(19) United States
(12) Patent Application Publication
(10) Pub. No.: US 2024/0350248 A1
(43) Pub. Date: Oct. 24, 2024

(54) PROTECTIVE FEMALE HYGIENE DEVICE

(71) Applicant: Marquetta Lusk Howard, Maricopa, AZ (US)

(72) Inventor: Marquetta Lusk Howard, Maricopa, AZ (US)

(21) Appl. No.: 17/980,032

(22) Filed: Nov. 3, 2022

(51) Int. Cl. A61F 2/00

(52) U.S. Cl. CPC A61F 2/0009; A61F 2250/0059; A61F 2250/0078

(57) ABSTRACT

The present subject matter depicts a device for protecting the vulva and labia. The present subject matter is used in the context of waxing, hair removal, sunbathing, or any other scenario where the protection of female genitalia is desired.

### FIELD

The present invention relates to protective female hygiene device. The present invention particularly relates to a protective shield and cover for protecting the vulva and labia minora of a female.

### BACKGROUND

As the demand for bikini hair removal procedures increase, so does the demand for related products. There are many methods for removing unwanted pubic hair, including waxing, shaving, using a depilatory, and laser hair removal. Waxing, such as the “brazilian wax”, is a particularly popular choice, and is offered at many beauty salons and spas. Laser removal is becoming increasingly popular due to its success with permanently removing unwanted hair. Hair removal in a female's pubic region typically requires full exposure of the vulva area to ensure complete hair removal, placing the sensitive regions of the female genitalia at risk for damage and pain.

Although various devices for the protection of the female vaginal region have been provided in the prior art, such prior art devices known to us have been directed to either the needs of artists, models, dancers and the like, who are looking for a shield for female genitalia during performances, or to medical devices, such as may be used by patients undergoing some type of medical procedure. However, available versions of protective shields of which we are aware are not configured for use in an easily placed, strapless manner adaptable for use by sun bathers, nor have they been provided in inexpensive materials that are easily and inexpensively replaceable, i.e., having a disposable configuration. Depending on the situation, the needs of sunbathers, or users of indoor tanning beds, or of actors in adult films, for example and not by way of limitation, have 28 not been adequately addressed by prior art devices of which we are aware. Thus, there remains a significant and as yet unmet need for a simple, anatomically compatible disposable shield which can be easily and quickly installed for use in the protection of female genitalia, especially with respect to protection against ultraviolet exposures, so as to provide protection against sunburn of sensitive body parts.

### BRIEF DESCRIPTION OF THE FIGURES

FIG. 1 depicts a perspective, front view of a device according to some embodiments presently disclosed.

FIG. 2 depicts a rear view of the device shown in FIG. 1.

FIG. 3 depicts a side view of the device shown in FIG. 1.

FIG. 4 depicts a top view of the device shown in FIG. 1.

In the following description, like reference numbers are used to identify like elements. Furthermore, the drawings are intended to illustrate major features of exemplary embodiments in a diagrammatic manner. The drawings are not intended to depict every feature of every implementation nor relative dimensions of the depicted elements, and are not drawn to scale.

### DETAILED DESCRIPTION

In the following description, numerous specific details are set forth to clearly describe various specific embodiments disclosed herein. One skilled in the art, however, will understand that the presently claimed invention may be practiced without all of the specific details discussed below. In other instances, well known features have not been described so as not to obscure the invention.

As described herein, the term “removably coupled” and derivatives thereof shall be used to describe a situation wherein two or more objects are joined together in a non-permanent manner so as to allow the same objects to be repeatedly joined and separated.

Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings.

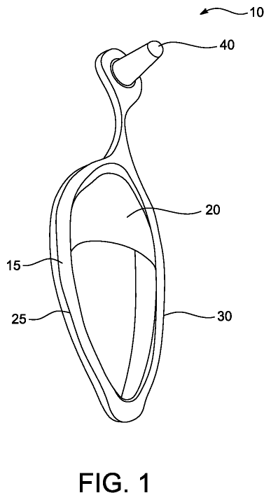

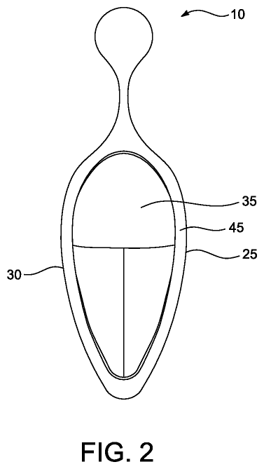

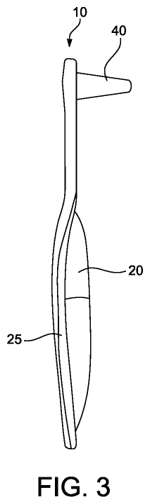

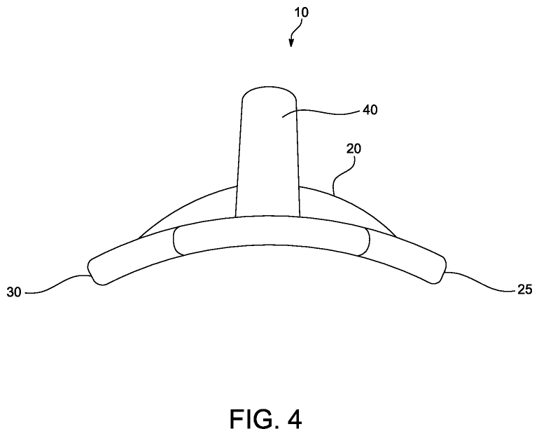

According to some embodiments, a protective female hygiene device 10 (shown in FIGS. 1-4) is presently disclosed. The presently disclosed protective female hygiene device (i.e. protective shield) 10 may be used for removal of hair in the female genital area. Such techniques may include, but are not limited to, waxing, laser removal, electrolysis removal, shaving, and use of depilatory creams, gels or sprays.

The device 10 may be used in protecting the female genital area, i.e., the vulva and the delicate membrane of the vaginal and urethral openings, the labia minora and all other very sensitive features. The device 10 may be shaped to fit over the entire labia minora of a human female, and may fit between the labia minora and the labia majora with the device 10 positioned around and over, and held in place by, the labia minora.

The presently disclosed protective female hygiene device (i.e. protective shield) 10 comprises a main body 15 shaped and adapted to be positioned so as to cover the labia minora and vulva of the female. The main body 15 may comprise a cap side 20 and two opposing lateral sides 25 and 30 extending from the cap and joined together at opposing ends of the cap to form a cavity side 35. The cap side 20 is positionable over the labia minora of the female with the lateral sides 25 and 30 fitting between the labia minor and labia major of the female such that the vulva and labia minora are covered by the main body 15 and held in place only by the natural contours of the labia minora. According to some embodiments, the cap side 20 is positioned opposite the cavity side 35.

The device 10 may also comprise a plug 40 as shown in FIGS. 1 and 3-4. According to some embodiments, the plug 40 may be inserted into anus to protect anus from chemicals used for removal of hair in the female genital area. According to some embodiments, the plug 40 may be inserted into anus to secure the device 10 in place during removal of hair in the female genital area.

During use, the device 10 may be positioned over the labia minora of the female with the lateral sides 25 and 30, fitting between the labia minora and labia majora of the female such that the vulva and labia minora are covered by the cap side 20, and such that the labia minora fits within the cavity side 35 of the device 10. The device 10 may be self held in place by the natural contours of the labia minora.

According to some embodiments, the outer edges of the lateral sides 25 and 30 are configured with a ridge formation 45 which provide additional gripping action According to some embodiments, the device 10 may comprise soft rubbery polymer material, which grips the labia minora of the user, and due to its non-breathable characteristics, may be installed over the labia minora so as to form a vacuum, which further assists in holding the device 10 in place when positioned in place over the labia minora via suction action. The soft rubbery polymer material may be, by way of illustration only and not limitation, an elastomer or ethylene-vinyl acetate (EVA). The soft rubbery polymer material may also be silicon, which disperses and reflects light energy so as to prevent penetration of light energy completely through the material when used for laser hair removal. The soft rubbery polymer material may also be made of other materials, such as rubber, plastic, vinyl, etc. According to some embodiments, the color of the device 10 is a color that substantially disperses and reflects, or blocks, light energy so as to prevent penetration of the light energy through the shield.

According to some embodiments, the device 10 comprises substantially oval shape to accommodate labia minora.

According to some embodiments, the cavity side 35 of the device 10 is formed to accommodate labia minora of different sizes and shapes that may exist because of the difference between the anatomies of females, especially on the upper aspect of the genital area, where some patients have a larger clitoris or labia minora. The device 10 may comprise the soft rubbery polymer material to provide for a comfortable fit regardless of the individual features of the patient. The presently disclosed device 10 may fit most patients so that the user's entire labia minora is covered. To accommodate different sizes of labia minora, the device 10 may be made in different sizes.

According to some embodiments, the main body 15 of the device 10 may be formed with indicator lines along the edges of the lateral sides 25 and/or 30 of the main body 15 to guide a user or practitioner in decreasing the depth of the cavity of the protective shield. In other words, the user or practitioner may use the indicator lines to cut the protective shield down to size to fit the user. The size selected size is specific to the user.

According to some embodiments, the retaining means is the implementation of the device 10 with a soft polymer material that naturally grips the inner surface of the protective shield through friction, as illustrated in FIG. 1-4. Other retaining means, such as an inward-formed ridge as shown in FIG. 1-4. Other retaining means may include mating grooves or ridges, clips, snaps, etc.

As described above, the protective shield is specifically contoured to fit over the woman's labia minora and held in place by custom fit inside the labia majora. When installed over the labia minora to remove as much air as possible, the protective shield may also stay in place due to suction action. This design allows the user or practitioner to stretch the labia majora in order to expose all hair for removal.

The unique fitted design of the protective shield with or without the outer cover is suited for placement by the user for home use or to wear to a hair removal practitioner's office or salon for personal cover. Although described herein with respect to use in hair removal procedures, the protective shield with or without the outer cover is also ideal for use in nude sunbathing and in surgical procedures performed near the sensitive vulva area.

While several illustrative embodiments of the invention have been shown and described, numerous variations and alternative embodiments will occur to those skilled in the art. Such variations and alternative embodiments are contemplated, and can be made without departing from the scope of the invention as defined in the appended claims.

As used in this specification and the appended claims, the singular forms “a,” “an,” and “the” include plural referents unless the content clearly dictates otherwise. The term “plurality” includes two or more referents unless the content clearly dictates otherwise.

Unless defined otherwise, all technical and scientific terms used herein have the same meaning as commonly understood by one of ordinary skill in the art to which the disclosure pertains.

The foregoing detailed description of exemplary and preferred embodiments is presented for purposes of illustration and disclosure in accordance with the requirements of the law. It is not intended to be exhaustive nor to limit the invention to the precise form(s) described, but only to enable others skilled in the art to understand how the invention may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom. Applicant has made this disclosure with respect to the current state of the art, but also contemplates advancements and that adaptations in the future may take into consideration of those advancements, namely in accordance with the then current state of the art. It is intended that the scope of the invention be defined by the Claims as written and equivalents as applicable. Reference to a claim element in the singular is not intended to mean “one and only one” unless explicitly so stated. Moreover, no element, component, nor method or process step in this disclosure is intended to be dedicated to the public regardless of whether the element, component, or step is explicitly recited in the claims. No claim element herein is to be construed under the provisions of 35 U.S.C. Sec. 112, sixth paragraph, unless the element is expressly recited using the phrase “means for . . . ” and no method or process step herein is to be construed under those provisions unless the step, or steps, are expressly recited using the phrase “step(s) for . . . ”

What is claimed is:

1. A protective shield and cover for protecting the vulva and labia wherein the main body 15 is shaped to protect the labia minora and vulva by the cap side 20, wherein the device 10 and lateral sides 25, 30 may be held in place by the natural contours of the labia, wherein the cavity 35 is shaped to accommodate different sizes of labia, wherein the plug 40 may be inserted into the anus as to protect from chemicals and to further secure the device 10.

2. The protective shield and cover for protecting the vulva and labia of claim 1, wherein the main body 15 is shaped to protect the labia minora and vulva by the cap side 20;

3. The protective shield and cover for protecting the vulva and labia of claim 1, wherein the device 10 and lateral sides 25, 30 may be held in place by the natural contours of the labia;

4. The protective shield and cover for protecting the vulva and labia of claim 1, wherein the cavity 35 is shaped to accommodate different sizes of labia;

5. The protective shield and cover for protecting the vulva and labia of claim 1, wherein the plug 40 may be inserted into the anus as to protect from chemicals and to further secure device 10.
